(19) United States
(12) Patent Application Publication
(10) Pub. No.: US 2017/0224746 A1
(43) Pub. Date: Aug. 10, 2017

(54) Nutritional Support Method For Health Issues

(71) Applicants: John Edgar Menear, Santa Cruz, CA (US); Joseph Ramaekers, Aptos, CA (US)

(72) Inventors: John Edgar Menear, Santa Cruz, CA (US); Joseph Ramaekers, Aptos, CA (US)

(73) Assignee: CortControl, Santa Cruz, CA (US)

(21) Appl. No.: 15/731,100

(22) Filed: Apr. 18, 2017

Related U.S. Application Data
(62) Division of application No. 14/544,274, filed on Dec. 17, 2014, now abandoned.
Application No. 14/544,274 is a continuation-in-part of application No. 13/843,581, filed on Mar. 15, 2013.
Application No. 13/843,581 is a continuation-in-part of application No. 13/718,319, filed on Dec. 18, 2012.
Application No. 13/718,319 is a continuation-in-part of application No. 12/631,745, filed on Dec. 4, 2009, now abandoned.
Application No. 12/631,745 is a continuation-in-part of application No. 11/494,464, filed on Jul. 28, 2006, now Pat. No. 7,511,920.

(51) Int. Cl. A61K 35/744; A23L 33/135; A61K 41/00; A23L 33/00

(52) U.S. Cl. CPC A61K 35/744; A23L 33/30; A23L 33/135; A23L 33/40; A61K 41/0004

(57) ABSTRACT

A method of improving the outcome or recovery for depression, diabetes, cardiovascular disease, Alzheimers progression, or breast cancer. This method consists of administering a food which includes transfer factor, lactic acid generating bacteria, and/or glucans in appropriate combinations and dosage levels. The food reduces cortisol levels, enhances the immune system, and balances hormones and cytokines. Dosage amounts are dependent on client weight. Consumption frequency and dosage may be adjusted. Typically, consumption of the food is done in combination with established medical treatments.

### CROSS-REFERENCE TO RELATED APPLICATIONS

This application is a divisional continuation of non-provisional application U.S. Ser. No. 14/544,274 filed Dec. 17, 2014. Application U.S. Ser. No. 14/544,274 is a continuation-in-part of non-provisional application U.S. Ser. No. 13/843,581, filed Mar. 15, 2013, which is a continuation-in-part of application Ser. No. 13/718,319, filed on Dec. 18, 2012. Application Ser. No. 13/718,319 is a continuation-in-part of application Ser. No 12/631,745, filed Dec. 4, 2009 and a continuation-in-part of application Ser. No 11/492,464, filed Jul. 24, 2006. The current application also claims priority to U.S. provisional application 61/964,100 filed Dec. 24, 2013.

### STATEMENT REGARDING FEDERALLY SPONSORED RESEARCH

Not Applicable

### REFERENCE TO A MICROFICHE APPENDIX

Not Applicable

### BACKGROUND OF THE INVENTION

Field of the Invention

This invention relates to the treatment of human disorders, which are correlated with an excess of cortisol (corticosteroids). More specifically, this invention describes a method of feeding a medical food composition that mitigates multiple disorders by reducing a patient's cortisol level. Cortisol is the body's primary stress hormone, and stress is widely considered a disease factor by the medical community.

Description of Related Art

A combination of transfer factor, glucans and lactic acid generating bacteria has been shown to increase immunity and balance the endocrine system of mammals. It was further demonstrated in mammal studies that this combination lowers cortisol. This was further supported by success in treating Cushings Disease, which is defined by high cortisol.

Anecdotal studies show the connection between cortisol and the above combination. After consumption, athletes performed better during games and students performed better on tests. In both cases, reduced stress was reported as the reason for improved performance.

Cortisol output is initiated when pituitary gland secretions of ACTH stimulate the adrenal glands, which are the producer of glucocorticoid hormones in the zona fasciculata.

Cortisol is the primary human stress hormone. Health is adversely affected when chronically stressed. Stress (chemically indicated by cortisol) normally causes a surge in adrenal hormones like adrenaline that increase alertness. High cortisol makes it more difficult to relax and perform well—especially when levels remain high or rise and fall irregularly.

Frequent or constant stress can chronically elevate these hormone levels, resulting in a hyper-vigilant state accompanied by adrenalin.

Normally, cortisol exhibits a periodic rhythm: high in the morning; moderate in the afternoon; and low at night. Both the periodic rhythm and the absolute concentration are consequential to good health. For example, shift work throws off the body's circadian rhythm and may lead to health problems. Examples of health problems include depression, diabetes, cardiovascular disease, Alzheimers progression, and breast cancer.

In a preventative scenario, the combination of transfer factor, glucans and lactic acid generating bacteria is consumed prior to evident medical symptoms. This instant application is more concerned with supporting treatment after the condition becomes symptomatic.

Medications are commonly used. But, if high cortisol is an underlying factor, the effect is muted. A medication might treat symptoms, but leave the underlying issue intact.

Highly addictive benzodiazepines (Xanax, Ativan, or Klonopin) are the only class of pharmaceutical in current use capable of temporarily suppressing cortisol levels.

The above treatments are intrusive. A food based approach (alone or in combination with other therapies) is more desirable. The side effects of medical foods are far less than conventional therapies.

A food based solution is particularly appropriate in view of current attitudinal trends.

The consuming public now understands that foods possess more than basic nutrition (protein, carbohydrate, fat, etc.). For example, 95% of consumers agree that foods have health benefits that go beyond basic nutrition and may reduce the risk of disease. More than 50% of U.S. consumers believe that foods can replace the use of drugs.

The Federal Drug Administration acknowledges the trend of augmenting medications with foods. New categories of “Medical Foods” or “Authorized Suppleements” reflect this. Medical foods should be administered or monitored by a doctor, nutritionist, nurse, medical technician or equivalent health care professional. Medical foods are defined by the Federal Drug Administration in 21 CFR 101.9(j)(8). Supplement use is less restricted.

A food that reduces cortisol is needed as a stand-alone treatment for sleeplessness or as an improvement to conventional remedies.

### BRIEF SUMMARY OF THE INVENTION

Following is a condensed summary of the invention. By necessity, details are omitted to simply state the essence of the invention. Omitted details within this section should not be construed in a way that limits the scope of the invention.

The instant invention is a method of treating five different conditions with a single method and single formulation that reduces human cortisol levels, simultaneously builds the immune system, and balances hormone (and cytokine) levels.

This method is directed to people with the five different conditions, whether high cortisol is present or not. If high cortisol is present, the disclosed treatment method is particularly effective. If high cortisol is not present, the disclosed treatment method remains effective due to immune system building and hormone (and cytokine) balancing.

This invention does not claim to address all disorders. This invention addresses only the five conditions cited.

The formulation, which is tied to the invented method, contains at least transfer factor and lactic acid generating bacteria. Any medical food or supplement composition that includes transfer factor and lactic acid generating bacteria is within the scope of this method invention. A formulation that does not include transfer factor and lactic acid generating bacteria is outside the scope of this method. Other components, in conjunction with transfer factor and lactic acid generating bacteria, may be used and remain within the scope of this method.

Addition of glucans further enhances the composition effectiveness.

The cortisol-reducing property of the composition was first measured during controlled cattle studies. Diseased cattle showed significant health improvement and reduced cortisol, relative to a control group. During the cattle studies, blood cortisol was measured over time as a chemical marker. As the animals improved, decreased cortisol levels were observed. Cortisol levels in the test cattle (calves) decreased from 34.3 to 21.5 ng/ml.

Although this data involves cattle, cross-over charts indicate that the biochemical response of cattle and humans are similar. Further, transfer factor is known to function across species. For instance, bovine transfer factor builds immunity on dogs and humans, and ovum transfer factor builds immunity on cattle and humans.

A cortisol-lowering response in humans is observable via dietary inclusion of transfer factor and lactic acid generating bacteria. Self-reported stress levels tend to drop after 9 days consumption.

Self-reported stress levels tend to drop after 9 days consumption. Over time, consumption should reduce the incidence of depression, diabetes, cardiovascular disease, Alzheimers progression, and breast cancer.

The immune building action of transfer factor and lactic acid generating bacteria further supports health by lowering physical stresses within the human body.

This application is not limited by any specific mechanism that explains how cortisol is lowered or how immune function is increased or how hormones are balanced. Claims are based on observation rather than theory.

Glucans may be added to the combination of transfer factor and lactic acid generating bacteria to increase effectiveness. Other useful additives include gamma amino butyric acid (GABA). GABA is the brain's primary inhibitory (i.e., calming) neurotransmitter. It neutralizes adrenaline as a primary function. Serotonin and melatonin are also appropriate additives in some cases.

It would be obvious to a person of ordinary skill that other forms of administration—such as injection, capsule swallowing or intravenous administration—would lead to the same result.

Objects of this invention include some or all of the following: (1) support diabetes treatments, (2) support treatments for depression, (3) support treatments for early stage Alzheimers Disease, (4) support treatments for breast cancer, and (5) support treatments for cardiovascular disease.

### BRIEF DESCRIPTION OF THE SEVERAL VIEWS OF THE DRAWINGS

FIG. 1 shows evening effect of the invented medical food from a cattle study. Note that cortisol levels decreased.

FIG. 2 shows morning effect of the invented medical food from a cattle study. Note that cortisol levels decreased.

FIG. 3 is a black-and-white picture that shows a calf with severe warts.

FIG. 4 shows the same calf from FIG. 3 after consumption of the invented food by the invented method.

### DETAILED DESCRIPTION OF THE INVENTION

A composition of transfer factor and lactic acid generating bacteria was patented by Joseph Ramaekers (a current joint inventor). Refer to U.S. Pat. No. 6,962,718, claim 6, issued Nov. 8, 2005, which recites, “A formulation comprising pharmaceutically acceptable transfer factor and a pharmaceutically acceptable lactic acid generating bacteria wherein the amount of said transfer factor is from 10 mg to 10,000 mg per ounce of formulation”. In a preferred formulation, glucans are added to the transfer factor and lactic acid generating bacteria.

The formulation of transfer factor, glucans, and lactic acid generating bacteria is used in conjunction with the invented method.

The invented method comprises feeding the formulation to a human who has been diagnosed with depression, diabetes, cardiovascular disease, Alzheimers progression, or breast cancer. Consumption begins 3-60 days prior to the onset of medical treatment. The 3-60 day period is based on animal studies cited in Patent - - - . Human response time parallels non-human mammal response time. The purpose is to build the body's self-healing capacity prior to medical intervention.

Self-healing capacity is defined as (1) increasing the immune response, (2) lowering cortisol levels, or (3) balancing hormones and cytokines.

This composition has been used successfully as a medicinal food in the veterinary field for a variety of animal diseases. For example, feedlot cattle showed decreased mortality. Also, mammal fertility was increased. Cushings disease was mitigated.

One of the benefits of this medical food formulation is cortisol reduction. A second benefit is immune system building. A third is hormone balancing.

Short periods of elevated cortisol are useful. But persistent high levels of cortisol degrade health and lead to disease.

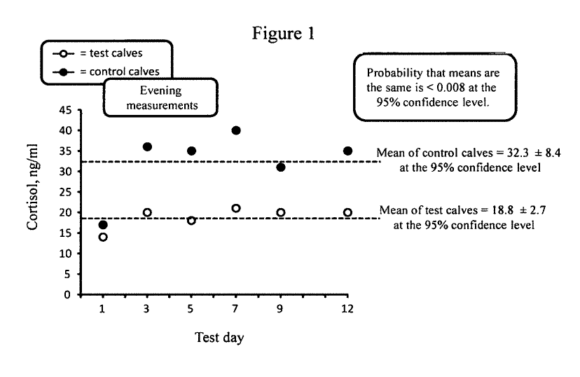

FIG. 1 summarizes the evening cortisol reduction (test versus control) for a calf study over a 12-day period. More current tests indicate that humans experience a similar evening cortisol reduction after consuming the medicinal food.

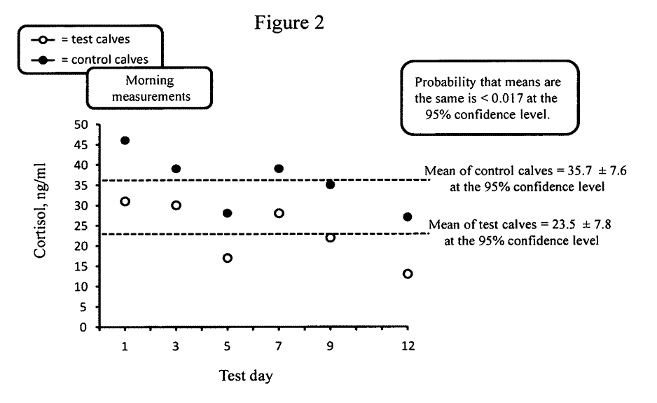

FIG. 2 summarizes the morning cortisol reduction (test versus control) for a calf study over a 12-day period. Humans experience a similar cortisol reduction after consuming the medicinal food.

Laboratory tests have demonstrated the immune building properties of combining transfer factor, lactic acid generating bacteria, and glucans. This food combination can increase the immune system more than five times the baseline level. The metric to demonstrate this five-fold increase was killer T-cell count.

This method-of-use invention is designed for five different health problems. The same method and formulation apply to each health condition. An ingested medicinal food utilizes the human cortisol-reducing, immune-building, and hormone balancing properties of that medicinal food to help multiple disorders.

Health disorders have multiple causes. Not all diseases respond to the consumption of transfer factor, glucans, and lactic acid generating bacteria. But depression, diabetes, cardiovascular disease, Alzheimers progression, or breast cancer do respond. And if a person has both high cortisol plus depression, diabetes, cardiovascular disease, Alzheimers progression, or breast cancer—that person is particularly amenable to this current method.

Components of the medical food include transfer factor and lactic acid generating bacteria. Glucans may be added to increase effectiveness. Following is a description of each component.

Transfer factor is produced by leucocytes and lymphocytes. Transfer factor comprises small water soluble polypeptides of about 44 amino acids that stimulate or transfer cell mediated immunity from one individual to another.

The properties, characteristics and processes for obtaining transfer factor are discussed in U.S. Pat. Nos. 4,816,563; 5,080,895; 5,840,700, 5,883,224 and 6,468,534, the contents of which are hereby incorporated by reference into the present application.

Alternative sources of transfer factor include avian transfer factor, ova transfer factor, and colostrum from goats, pigs, horses and humans. This listing is not complete. In addition, combinations of transfer factors from any number of sources may be used in human health formulations.

In certain embodiments, a significant fraction of transfer factor has a molecular weight of less than 10,000 Daltons. Transfer factor may be derived from colostrum, but it is considerably different from colostrum. It takes approximately 1000 grams of colostrum to produce 1 gram of the transfer factor recited in this application.

Transfer factor is commercially available, and known to be safe. Transfer factor is included in the Physician's Desk Reference.

Transfer factor contains both inducer and suppressor fractions.

The inducer fraction of transfer factor links the immune cells with an antigen-binding site, thereby increasing their reactivity to an antigenic stimulus. This is advantageous when a higher immune response is required. CortControl's enhanced vaccine response (a separate benefit, not discussed here) arises from an inducer fraction. The inducer effects of transfer factor are documented by several researchers.

Transfer factor's suppressor fraction blocks the response of the T-cells and signals a down-regulation of the immune response. This is useful in allergic or autoimmune conditions. Animal studies demonstrate improvement for cases where the immune system is over-active. This arises from suppressor fraction. Cortesini, R.et.al. demonstrated that CD8+CD28−Ts represent a unique subset of regulatory cells within transfer factor that initiates a suppressive loop. Filaci supported Cortesini's work, and related auto-immune disease the absence of CD8+ suppressor T lymphocytes. Filaci further found that “CD8+ Ts can be generated in vitro from CD8+CD28−T lymphocytes. A key role in their generation is played by monocytes that secrete interleukin-10 (IL-10) after granulocyte macrophage-colony-stimulating factor (GM-CSF) stimulation.”

Helper lymphocytes develop along two lines of cell populations: TH1 and TH2.

TH1 and TH2 cells perform different functions and produce different cytokines. Cytokines are proteins that function as messenger molecules. Transfer factors from both lymphocytes lines are represented in transfer factor.

TH1 cells modulate cell-mediated immunity. TH1 cells produce the following cytokines: IL-2, IFN-gamma, and TNF-alpha.

TH2 cells modulate humoral immunity (antibody production). TH2 cells produce the following cytokines: IL-4, IL-5, IL-6, IL-10, and IL-13.

Cytokines are functionally similar to hormones, but are not associated with a specific gland. Cytokines appear to operate as a key and template. Stereochemistry is important. Attachment of a cytokine on an immune cell receptor will start a specific immune signal within the target cell. Signals translate into direct action, increase the production of antibodies against an invading virus, or initiate the production of other cytokines to propagate the signal.

Transfer factor can change TH1/TH2 predominance conditions within 48 hours. This observation begs the question, “How can Transfer Factor change a predominantly TH-2 Immune System to a predominant TH-1 System so rapidly?” (The hypothesis of new TH1 cells was dismissed early because it takes 10-14 days to mature new TH1 cells.)

The answer is “conversion by eomesodermin,” which was studied in 2003. In that study, already-differentiated TH2 cells were converted to TH1 cells. Eomesodermin is a T-box transcription factor related to T-bet and already characterized as a key regulator of mesodermal differentiation. Eomesodermin was specifically up-regulated in activated CD8+ cells [one of transfer factor's actions].

Conversion of TH2 to TH1 cells is applicable to viral vaccines and malaria because both conditions tend to be TH2 dominated. In the opposite direction, conversion of TH1 to TH2 is advantageous for Rheumatoid arthritis or Crohn's disease.

Good health requires a balance of TH1 and TH2. The invented food, partly through transfer factor, assures that balance. The TH1/TH2 balance supports a healthy body that can take advantage of medical treatment for depression, diabetes, cardiovascular disease, Alzheimers progression, and breast cancer.

Lactic acid generating bacteria is a component of the food used in this method, and is GRAS (generally recognized as safe). Lactic acid generating bacteria support digestion and brain health. Lactic acid generating bacteria provide healthful effects that are found in non-pasteurized sauer kraut and cod liver oil. Within the intestinal tract, lactic acid generating bacteria are beneficial. It has been estimated that 80% of human health depends on beneficial intestinal bacteria. Intestinal bacteria account for 90% of the total human cell count.

Lactic acid generating bacteria is only one component of the formulation used in the invented method, but it is an important inclusion.

A human body becomes stressed by poor digestion, and cortisol levels will increase to reflect that stress. Stated differently, lactic acid generating bacteria helps reduce cortisol via improved digestion.

Metabolic interactions between the ecosystem and host affect health and well-being. Host cells are grossly outnumbered. A human body consists of 15-70 trillion cells; for each human cell, there are 9 microflora or bacteria cells. A symbiotic relationship exists. A healthy gastrointestinal tract parallels a healthy host. In contrast, imbalances in the microbial community are implicated in diseases. For instance, bacterial compositions differ between children who develop atopic diseases and those who do not, and between children from countries with a high or low incidence of atopic disease.

The ecosystem within the GI tract is responsible for other essential processes including: immune system regulation, vitamin bioavailability, sleep cycle, brain health, cytokine production, and vitamin K production.

Lactic acid generating bacteria compete against or “antagonize” an array of pathogens including Escherichia coli (E. coli, the cause of “Montezuma's Revenge”), Staphylococcus aureus and Salmonella (common causes of food poisoning), Candida albicans (yeast infections and syndromes), and other pathogens such as Shigella, Clostridium, Listeria, and Helicobacter species. This directly impacts the body's capacity to heal.

Glucans (polysaccharides) are known to support the immune system. When combined with transfer factor and lactic acid generating bacteria, a synergy is created. The combined effect on healing capacity is greater than the effect predicted from summing the individual components. Glucans may be present as natural or hybrid mushrooms.

An important function of beta-glucans is immuno-modulation. A century and a half of research ^{[4]}has shown that beta glucans act as immuno-modulating agents, meaning they trigger a cascade of events that help regulate the immune system, making it more efficient.

Beta glucans effectively bind and activate specific innate immune cells including T-cells, NK (natural killer) cells, and macrophages. The ability of beta glucans to modify the attack of immune cells on invasive agents supports an efficient and stronger immune response, while causing minimal damage to the rest of the body. “Minimal damage” includes avoidance of over-stimulation, which leads to autoimmune diseases.

Immuno-suppression is observed in people with stress-related diseases, such as coronary disease. Under stress, the number of macrophages available are reduced and unable to participate in the immune cascade. This causes even deeper immuno-suppression. Beta-1,3/1,6-glucan has been shown to nutritionally potentiate and activate macrophage cells which may assist in countering these effects.

Beta-glucans are not highly soluble, and particles that pass through the intestines are relatively large. Enterocytes facilitate the transportation of β(1,3)-glucans and similar compounds across the intestinal wall into the lymph, where they begin to interact with macrophages to activate immune function. Large beta-glucans are broken down in the macrophage, and made available to other immune cells.

Beta-glucan is an opsonin. An opsonin is any molecule that enhances phagocytosis by marking an antigen for an immune attack or marking dead cells for recycling. Opsonization (also, opsonisation) is the molecular mechanism whereby molecules, microbes, or apoptotic cells are chemically modified to have stronger interactions with—to be more “delicious” to—cell surface receptors on phagocytes and NK cells. With the antigen coated in opsonins, binding to immune cells is greatly enhanced.

A coating of beta-glucans on an invader makes the invader more susceptible to leukocyte removal. Binding reaction is greatly enhanced. Opsonin translates as “to prepare for eating”. Opsonization further mediates phagocytosis via signal cascades from cell surface receptors.

A medicinal food composition may be augmented with additional additives. Example additives are minerals, probiotics, prebiotics, dimethyl glycine, ascorbic acid, Vitamin A, Vitamin B1, Vitamin B2, Vitamin B12, Vitamin D3, Vitamin E, dipotassium phosphate, potassium chloride, magnesium sulfate, calcium pantothenate, minerals, antioxidants, amino acids, nutraceuticals, inositol hexaphosphate (Ip6), mannans, olive leaf extract, and phytosterols. In certain preferred embodiments, mannans are derived from Aloe Vera. In certain preferred embodiments, phytosterols may be derived from soya bean.

Probiotic additives include, but are not limited to B. subtlis, B. longum, B. thermophilium, B. coagulans, E. faecium, S. cerevisia, L. casei, L. plantarum, Pediococccus acidilacticii, Kluyveromyces marxianus fragillis, and combinations thereof.

The above listings do not include all possible additives. The food compositions may also include one or more of the following: carrier proteins such as serum albumin; buffers such as sodium acetate; fillers such as microcrystalline cellulose, lactose, corn and other starches; binding agents; sweeteners and other flavoring agents; coloring agents; and polyethylene glycol. Additives are well known in the art, and are used in a variety of formulations.

Neurotransmitter precursors such as GABA, tryptophan, and/or melatonin may he added to the medicinal food formulation.

The relative proportion of transfer factor, lactic acid generating bacteria, and glucans within the composition may vary widely. And dosage levels can be adjusted. The reasons for the wide ranges are (1) that humans can create cortisol by imagining threatening scenarios, and (2) that different stages of treatment are involved.

However, some reasonable weight ranges for transfer factor are 0.05-50 mg per pound of body weight. Reasonable weight ranges for lactic acid generating bacteria are 0.47-10 mg per pound of body weight. This is based on a nominal live count of 2.5×10⁹ CFU/Ounce. Reasonable weight ranges for glucans are 0.1-10 mg per pound of body weight.

A method of using the food composition may have some or all of the following steps:
(1) determine that depression, diabetes, cardiovascular disease, Alzheimers progression, or breast cancer is present,
(2) select the correct proportion of transfer factor, lactic acid generating bacteria, and glucans,
(3) choose the correct dosage level,
(4) select a feeding frequency between five times per day and once per week,
(5) begin consumption of the medical food 3-60 days before medical intervention begins,
(7) continue consumption until throughout the medical treatment, and
(8) consume the medical food on a maintenance basis after treatment as needed. This may be done at the treatment dosage or at a reduced dosage.

For some patients, transfer factor, lactic acid generating bacteria, and glucans are taken together. For other patients, transfer factor, lactic acid generating bacteria, and glucans are taken at different times during the day or week. Component separation and consumption at different times are within the scope of this invention. Separate consumption and was recited in Ramaekers' U.S. Publication 20070128253, which benefits this application. The human body performs the mixing.

Other stress hormones may also be lowered by consuming transfer factor, lactic acid generating bacteria, and/or glucans. Hence, other stress hormones may be monitored.

The physiology between animals and humans is similar. But methods of treatment differ for humans from animals. Two differences involve (1) treatment durations and (2) feeding frequency. Humans typically have a wider range of feeding frequencies, and longer treatment durations than livestock. This reflects the underlying treatment guidelines.

For livestock, the guideline is economic. Feeding is operationally fixed. Changing the feeding frequency affects a rancher's profit.

For humans, the guideline is effectiveness. Cortisol levels increase in response to stressful thoughts, and subside with calming thoughts. Hence, longer treatment periods for humans are often used, relative to livestock. Several small sleep improvement food portions per day—rather than 1 large portion per day—are not difficult for humans to arrange. Consumption frequency may vary between five times per day and once per week.

Hence, method steps in this application are different from animal applications for which filing benefit is claimed. Human patients respond to the invented sleep improvement food in different time frames. Treatment periods vary.

Periodically measuring cortisol during treatment is recommended. Based on periodic cortisol measurement, dosages, frequency of feeding, and component proportions are optimized. Without measurement, the treatment program cannot be exact. With measurement, treatment aspects can be better refined.

Disorders frequently overlap with other medical problems. And some of those other medical problems are also improved with a combination of transfer factor and lactic acid generating bacteria. Again, cortisol reduction, hormone/cytokine balance, and immune building are active.

Depression correlates with an excess of cortisol. With or without high cortisol, depression responds well to a medical food created from transfer factor, lactic acid generating bacteria, and/or glucans. Interestingly, more serotonin (known for mood and depression activity) is found in the intestines than in the brain.

In the United States, an estimated 6.7% of the U.S. population suffers from depression. This corresponds to roughly 21 million people.

The Department of Psychiatry, University of California, San Francisco, Calif. performed studies linking cortisol to human depression. A total of seven studies comparing plasma or cortisol responses to psychological stressors in clinically depressed (MDD) and non-depressed (ND) individuals (N=196: 98 MDD, 98 ND; 83 men, 113 women; mean age=40 years) were included. Sample size-adjusted effect sizes (Cohen's d statistic) were calculated and averaged across baseline (before stressor onset), stress (stressor onset up to 25 min after stressor offset), and recovery (more than 25 min after stressor offset) periods. Overall, MDD and ND individuals exhibited similar baseline and stress cortisol levels, but MDD patients had much higher cortisol levels during the recovery period than their ND counterparts. There was also a significant time of day effect. Afternoon studies were more likely to reveal higher baseline cortisol levels, blunted stress reactivity, and impaired recovery in MDD patients. This blunted reactivity-impaired recovery pattern observed among the afternoon studies was most pronounced in studies with older and more severely depressed patients.

Note that depressed clients are often treated with serotonin supplementation. But more serotonin is produced in the gut than in the brain when the gut bacteria are healthy. The lactic acid bacteria support gut health. Supplementation with lactic acid bacteria has been reported to help depression more than serotonin supplementation. Again, recovery outcome and recovery rates are improved by the invented food and method.

The method of use for depression is the same as for diabetes, cardiovascular disease, Alzheimers progression, or breast cancer. Again, the method is consumption of the formulation 3-60 days before standard medical or mental treatments begin.

Consumption may continue during and after treatments.

A second example of a health issue that frequently occurs in conjunction with cortisol is diabetes. Some categories of diabetes also correlate with high cortisol.

Prolonged elevation of cortisol causes diabetic-level blood sugar. It does so by increasing insulin resistance in the body. Insulin is the hormone that causes the body to uptake sugar from the blood. The body then stores the sugar in various tissues, mainly the liver and in fat tissue. With elevated cortisol, your body doesn't respond as well to the insulin signal. Thus, there is less uptake of sugar from the blood, leaving more sugar in the blood. This high sugar level is seen in patients with Type II diabetes mellitus.

In the United States, 25.8 million children and adults—8.3% of the population—have diabetes. Deaths attributed to diabetic causes are roughly 69,000 per year.

The combination of transfer factor, lactic acid generating bacteria, and/or glucans has been shown to lower cortisol. The important effect is that the rate of blood sugar uptake is restored, and blood sugar levels return to a normal range.

Again, the method of treatment remains the same. The medical food composition remains within the boundaries of this disclosure: (1) transfer factor and lactic acid generating bacteria, or (2) transfer factor, lactic acid generating bacteria, and glucans. Feeding dosages and frequencies are the same.

A third example of a health problem that responds to the invented method is cardiovascular disease. Patients with the highest cortisol levels were five times as likely to die of heart attack, stroke or other cardiovascular causes over a period of six years. Cardiovascular disease (CVD) is the leading cause of death (roughly 600,000 per year) in the United States, and is responsible for 17% of national health expenditures. As the population ages, these costs are expected to increase substantially.

Researchers found that hair cortisol levels were a more important predictor of heart attack risk than established risk factors like high blood pressure and cholesterol.

In the cardiovascular case, the invented food and method affects outcome by reducing cortisol levels. The outcome and recovery times of medical treatments are enhanced.

The method to improve cardiovascular patients remains the same: consumption of the medicinal food 3-60 days before formal medical treatment. The goal is to advantage the body to benefit from the formal medical treatment.

A fourth example of a health issue that responds to the medicinal food is Alzheimer's disease. In the early stages of Alzheimer's disease, cortisol reduction can slow the progression. The earlier cortisol reduction begins, the better the prognosis.

The U.S. incidence rate of Alzheimers is 5.2 million, and growing as the population ages. It is a particularly cruel disease.

Over the long term, elevated cortisol kills brain cells (this is widely accepted). The worst part is that the hippocampus is the part of the brain most affected. The hippocampus serves two important roles. New memories are formed in the hippocampus, and the hippocampus limits the generation of more cortisol.

Dr. Dharma Khalsa describes how some older people lose 20-25% of the hippocampus cells. Hence, the hippocampus can no longer function as the cortisol feedback control circuit. Cortisol production builds, more hippocampus cells are lost, even more cortisol is produced, etc. A degenerative cascade develops. Dr. Robert Sapolsky at Stanford University confirmed Dr. Khalsa's scenario.

When combined with currently accepted medical practices, the Alzheimers progression is slowed because the cortisol-and-brain-damage scenario is lessened. The destructive cycle is broken, and progression is arrested. Quality of life is extended.

Another issue that responds to the invented method is breast cancer. In the sense that cancer is a case of auto-immune dysfunction, the onset and development could have been helped. In addition, there are several people who survived surgery and chemotherapy, but never felt fully recovered. After 2 years of a tepid unsatisfactory recovery, 9 days of the invented food with the invented method felt like a robust recovery. This was self-reported. The woman in this story continues to consume the invented food daily. Several human survivors report that consuming the food formulation allows them to live a normal life; energy level motivation increases are self-reported. Immune buildup appears related since the frequency of colds and flus has dropped.

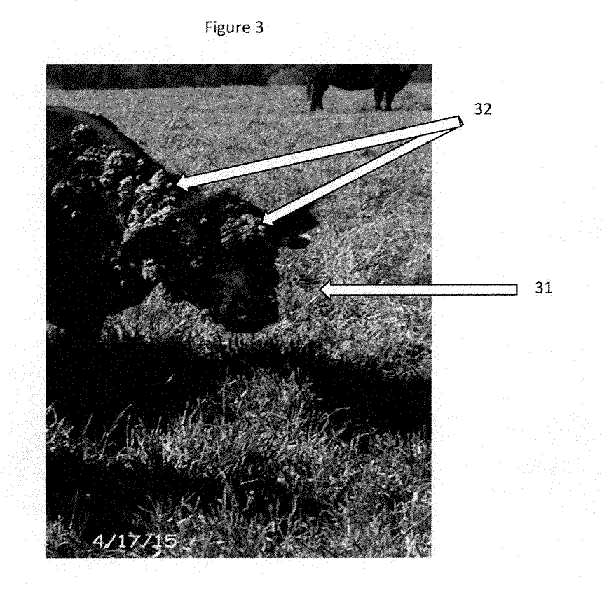

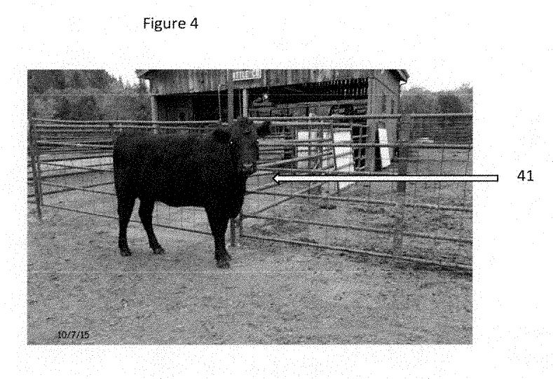

The effect on cancer spread has been studied in animals. FIG. 3 shows a calf 31 with severe warts 32. FIG. 4 the same calf 41 after consuming the medicinal food with the invented method. The after picture shows no warts remaining. From the medicinal food perspective, the warts arose from an auto-immune response. Cytokines generated from all three components of the treatment food were rebalanced, and the auto-immune condition no longer existed. Note that a “cure” is not being claimed. Instead, the calf's body was re-equilibrated to a normal condition.

An estimated 232,340 new cases of invasive breast cancer and 39,620 breast cancer deaths were expected to occur among U.S. women in 2013. About eight in ten breast cases and nearly nine in ten breast cancer deaths will occur among women aged 50 years and older.

Current medical treatments for breast cancer are stressful in themselves. Increased cortisol levels are expected. Women with advanced breast cancer who have abnormal daytime levels of cortisol are significantly more likely to die sooner than patients with normal levels of the hormone.

The medical food based on transfer factor, lactic acid generating bacteria, and/or glucans is a useful complement to medical treatment. As cortisol is reduced, health improves. And an improved immune system makes it easier to recover from chemotherapy, radiation, and surgery.

The medical food composition, dosage levels, and feeding frequencies remain are the same for all five conditions cited in this application.

Whe invention claimed is:

1. A method for improving the outcome or recovery rate of medical or psychiatric treatment for a human disease condition, relative to the outcome or recovery rate of medical or psychiatric treatment without the method,
wherein the disease condition is any one chosen from a group consisting of depression,
diabetes, cardiovascular disease, Alzheimers progression, and breast cancer, comprising:
providing a formulation of transfer factor, glucans and lactic acid generating bacteria to the human for consumption,
wherein said transfer factor includes polypeptides with a molecular weight below 10,000 Daltons;
choosing the dosage of the formulation based on the human's body weight; and
beginning the consumption 3-60 days before beginning the medical treatment.

2. The method of claim 1 wherein consumption frequency is between five times per day and once per week.

3. The method of claim 1 wherein said glucans are derived from natural or hybrid mushrooms.

4. The method of claim 1 further comprising:
adjusting said dosage levels during medical treatment.

5. The method of claim 1 further comprising:
changing the relative proportions of transfer factor, lactic acid generating bacteria, and glucans included within each said dosage.

6. The method of claim 1 wherein said transfer factor in each said dosage is present at 0.05 to 50 mg per pound of human body weight.

7. The food-based method of claim 1 wherein said lactic acid generating bacteria in each said dosage is present at 0.47 to 10 mg per pound of human body weight.

8. The method of claim 1 wherein depression is treated by a medically accepted procedure.

9. The method of claim 1 wherein diabetes is treated by a medically accepted procedure.

10. The method of claim 1 wherein cardiovascular disease is treated by a medically accepted procedure.

11. The method of claim 1 wherein Alzheimers progression is treated by a medically accepted procedure.

12. The method of claim 1 wherein breast cancer is treated by a medically accepted procedure.

13. The method of claim 1 wherein cortisol levels are a contributing factor to the symptoms of depression, diabetes, cardiovascular disease, Alzheimers progression, and breast cancer.

14. method of claim 1 wherein cytokines within transfer factor or produced by transfer factor affect the TH1 and TH2 balance.

15. The method of claim 1 wherein the glucans promote health by acting as an opsonin for overall pathogen removal.
